(19) United States
(12) Patent Application Publication
(10) Pub. No.: US 2025/0000335 A1
(43) Pub. Date: Jan. 2, 2025

(54) POSITIONING AID AND METHOD TO FEED TOOLS INTO CHANNELS OF A FLEXIBLE ENDOSCOPE THROUGH CONTROL HANDLE'S AIR/WATER AND SUCTION VALVE CYLINDER PORTS

(71) Applicant: American Sterilizer Company, Mentor, OH (US)

(72) Inventors: Nancy A. ROBINSON, Fernandina Beach, FL (US); Michelle M. NARANDZIC, Lakewood, OH (US); Elizabeth LESZCZ, Lakewood, OH (US)

(73) Assignee: American Sterilizer Company, Mentor, OH (US)

(21) Appl. No.: 18/710,470

(22) PCT Filed: Nov. 17, 2022
(86) PCT No.: PCT/US2022/050199

§ 371 Date: May 15, 2024

Related U.S. Application Data
(60) Provisional application No. 63/290,695, filed on Dec. 17, 2021.

(51) Int. Cl. A61B 1/00; A61B 90/00

(52) U.S. Cl. CPC A61B 1/00133; A61B 1/00066; A61B 90/08; A61B 2090/0807

(57) ABSTRACT

A positioning aid that is attachable to a handle of an endoscope control handle having air/water and suction valve cylinder ports and operable to guide a tool into at least one of the air/water or suction valve ports has a body for coupling to the endoscope control handle, the body including at least one guide port. The guide port includes an input port for receiving an instrument to be inserted into the air/water or suction valve cylinder ports, an output port configured to correspond with one of the air/water or suction valve cylinder ports, and a guide channel coupled between the input port and the output port.

### FIELD OF THE INVENTION

The present invention relates generally to cleaning accessories for medical instruments and, more particularly, to a device and method for inserting tools into channels of a medical instrument.

### BACKGROUND OF THE INVENTION

Unlike instrument and suction channels of a flexible endoscope, the air, water, and balloon channels of a flexible endoscope are not designed to allow tools (e.g., a long tube, wire, or flexible cord, for example a cleaning brush or swab) to be inserted therein. Such tools are used for a variety of purposes such as, for example, introducing a biological challenge (microorganisms in a soil) along a length of the channel or at the center of the channel segment, to clean or dry the channel segment, or to verify if the channel segment is clean or dry.

To feed a tool into the air, water, or balloon channel systems of a flexible endoscope, the user pre-bends the tool to case insertion into one of the internal openings (up to 6) present in the valve cylinder port. Next, the user attempts to introduce the pre-bent tool into one of the internal openings.

FIG. 1 diametrically illustrates a flexible endoscope control handle 10 with an air/water cylinder port 12 and a suction cylinder port 14. With additional reference to FIG. 2, actual control handle valve cylinder ports of an ultrasound endoscope are shown having five openings in the air/water valve cylinder port. Three of the openings are connected to tubes 16 extending from a right side of the air/water valve cylinder port 12. Ultrasound flexible endoscopes also have a balloon channel 18 connected to the suction cylinder port 14 that is not designed to allow introduction of a cleaning brush (i.e., it is difficult to gain entry into the channel 18).

Due to the tight spacing and different exit angles for each channel of a port, it can be difficult to insert a tool into the channels. Typically, multiple attempts are required to successfully introduce the tool into the channels using the openings in valve cylinder ports. A busy reprocessing technician would become frustrated and impatient with the process. Thus, while it is possible for a trained individual (who has exceptional manual dexterity, hand eye coordination, and patience) to insert a tool into the channels, most endoscope reprocessing technicians are not able to easily insert tools through the valve cylinder ports into the air, water, and balloon channels.

With the increased emphasis on quality checks and competency training for endoscope reprocessing technicians, there is an unmet need to gain easy access to the air, water, and balloon channels to confirm cleanliness and dryness.

### SUMMARY OF THE INVENTION

In accordance with the invention, a positioning aid and method are provided to feed tools into channels of a flexible endoscope through the endoscope's control handle air/water and suction valve cylinder ports. The positioning aid in accordance with the invention can be affixed to the control handle at the valve cylinder ports and provides endoscope model-specific passageways to guide a tool into the air, water and/or balloon channel segments of the respective ports. The tool openings on the positioning aid are situated to allow easy access by a user and do not require pre-bending of the tool that is to be inserted into the endoscope. The positioning aid is affixed into the cylinder port in a singular orientation so that the pathway exits (i.e., the locations where the positioning aid pathways end on the side wall of the aid) are properly aligned with the port's channel segment openings, i.e., the positioning aid can only be attached in one orientation.

In a first embodiment a positioning aid in accordance with the invention includes one or more pathways formed by flexible tubing, where exterior openings of the tubing extend beyond a body of the positioning aid. A terminus of each pathway is fixed into the correct position at the side of the positioning aid body to allow a tool to be inserted into the channel segment opening located inside of the valve cylinder port.

In a second embodiment of the positioning aid, multiple pathways are formed in a solid positioning aid, e.g., a 3D printed block or the like, whereby exterior tool openings are disposed at a top of the positioning aid and pathway exits occur at the correct locations along the side of the positioning aid body to allow a tool to be inserted into the channel segment openings. The pathways can be either straight or designed in a helical pattern so that the approach to the cylinder opening is at a gentle angle that the tools can easily negotiate.

In a third embodiment, a hybrid version is formed from the combination of the first and second embodiments. The third embodiment includes flexible and solid pathways, whereby the two pathway types can create separate pathways or a pathway that is partially flexible and partially solid.

According to one aspect of the invention, a positioning aid attachable to a handle of an endoscope control handle having air/water and suction valve cylinder ports having at least one channel and operable to guide a tool into at least one channel of the air/water or suction valve ports, the positioning aid includes: a body for coupling to the endoscope control handle, the body including at least one guide port having an input port for receiving an instrument to be inserted into the at least one channel in the air/water or suction valve cylinder ports, an output port configured to correspond with an opening to the at least one channel in the air/water or suction valve cylinder ports, and a guide pathway coupled between the input port and the output port, wherein a transition of the guide pathway between the input port and the output port exhibits curvature in more than one plane.

According to another aspect of the invention, a positioning aid attachable to a handle of an endoscope control handle having air/water and suction valve cylinder ports having at least one channel and operable to guide a tool into at least one of the air/water or suction valve ports, the positioning aid includes: a body for coupling to the endoscope control handle, the body including at least one guide port having an input port for receiving an instrument to be inserted into the at least one channel in the air/water or suction valve cylinder ports, an output port configured to correspond an opening to the at least one channel in the air/water or suction valve cylinder ports, and a guide channel coupled between the input port and the output port, wherein at least one of an angle of incidence of the guide channel at the interface with the input port or an angle of incidence of the guide channel at the interface with the output port is less than 20 degrees.

In one embodiment, an angle between a channel opening of a valve cylinder port and a straight portion of a segment of the guide channel corresponding to the channel opening as the straight portion of the segment approaches the channel opening is between 0 degrees and 30 degrees.

In one embodiment, a bend radius of a curved section that joins a first straight portion of a first guide channel segment of the guide channel and a second straight portion of a second guide channel segment of the guide channel is greater than 0.1 inches.

In one embodiment, an angle between the first and second straight portions at a connection point at respective ends of the curved section is greater than 100 degrees.

In one embodiment, the body comprises a three-dimensional printed body.

In one embodiment, the at least one guide port is formed entirely within the body.

In one embodiment, the guide channel is formed to have a helical pattern.

In one embodiment, the input port comprises flexible tubing extending out from the body.

In one embodiment, a terminus of the flexible tubing is fixed to a sidewall of the body at the output port.

In one embodiment, the positioning aid further includes a coupler connecting the flexible tubing to the output port.

In one embodiment, the coupler comprises a brass tube insert.

In one embodiment, the flexible tubing comprises a tube formed from polytetrafluoroethylene.

In one embodiment, the body comprises a keyway that cooperates with the handle to enable attachment of the body in only one orientation with respect to the handle.

In one embodiment, the body comprises a stop that limits a depth of insertion of the body into the handle.

In one embodiment, the guide channel is color-coded to identify a size of the guide channel.

In one embodiment, the guide channel is color-coded to identify a port of the endoscope.

An advantage of the positioning aid in accordance with the invention is that tools do not need to be pre-bent by the user prior to insertion into the endoscope's channels. The pathways within the positioning aid guide the tools into the channel openings within the air/water or suction cylinder ports. This makes the tools easy to insert, as the pathways in the positioning aid direct the tools into a specific hidden internal channel segment opening within the valve cylinder ports. Further, the pathways provide support to the tool as it negotiates entry into the channel segment.

Another advantage is that little to no training is required to insert tools into the channels. This is due to the positioning aid pathway openings being easy to see and access from the exterior of the valve cylinder ports. Also, placement of the positioning aid is fool proof, as in accordance with an embodiment of the invention the positioning aid can only be affixed to the control handle in one orientation, ensuring the pathway exits are correctly located to feed the tool into the channel openings within the valve cylinder ports.

To the accomplishment of the foregoing and related ends, the invention, then, comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed. Other objects, advantages and novel features of the invention will become apparent from the following detailed description of the invention when considered in conjunction with the drawings.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a schematic illustration of a flexible endoscope control handle.

FIG. 2 shows the air/water and suction valve ports in a handle of a typical ultra sound flexible endoscope control handle which include balloon channels.

FIG. 3 is a cross-sectional view of a valve port that may be used in the handle of FIG. 1.

FIG. 4 illustrates exemplary handle that has a tight radius in certain channels within the handle.

FIGS. 5A-5C illustrate an exemplary positioning aid in accordance with an embodiment of the invention.

FIG. 6A illustrates an exemplary positioning aid in accordance with another embodiment of the invention.

FIG. 6B illustrates the positioning aid when inserted into an air/water valve cylinder port.

FIGS. 7A and 7B illustrate an exemplary positioning aid in accordance with yet another embodiment of the invention.

FIG. 8 illustrates an exemplary positioning aid in accordance with yet another embodiment of the invention.

FIGS. 9A-9B illustrate three-dimensional renderings of the positioning aid of FIG. 6A.

FIG. 9C illustrates a three-dimensional rendering of the positioning aid of FIG. 7A.

FIG. 9D is a front plane section of a portion of the positioning aid in accordance with the invention showing two openings in a front plane.

FIG. 10 is a schematic diagram illustrating a bend angle, bend radius and angle of attack of an exemplary guide pathway in accordance with the invention.

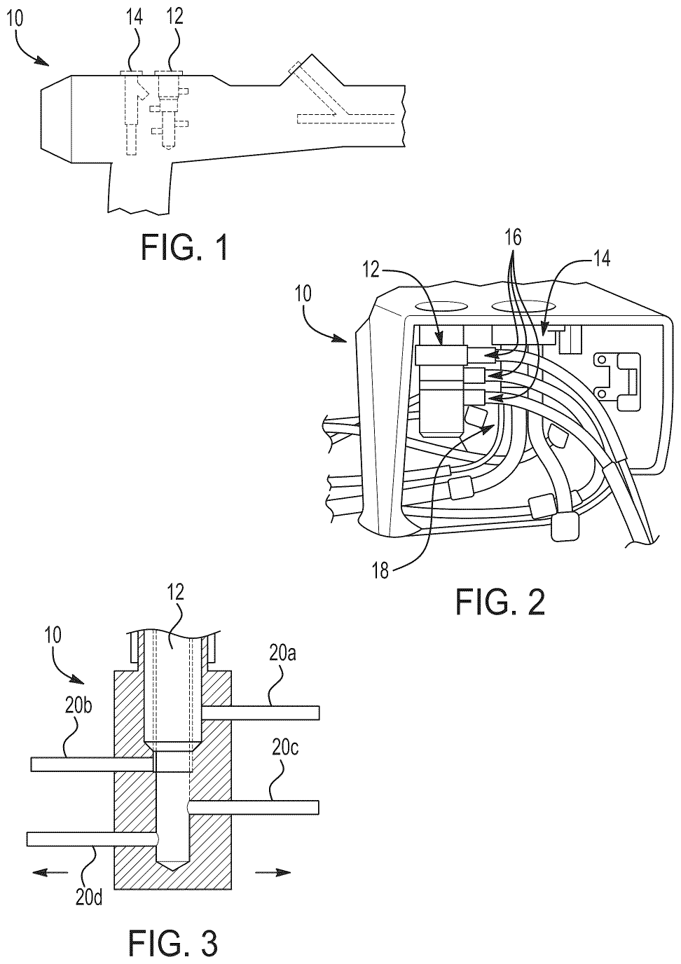

### DETAILED DESCRIPTION OF PREFERRED EMBODIMENT

Embodiments of the present invention will now be described with reference to the drawings, wherein like reference numerals are used to refer to like elements throughout. It will be understood that the figures are not necessarily to scale.

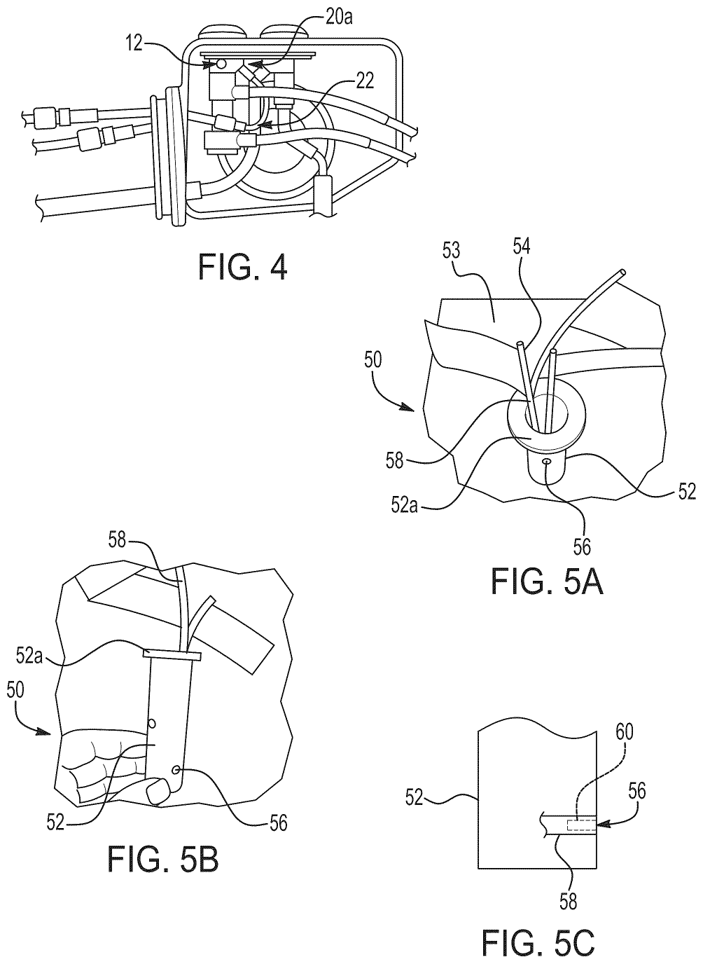

Referring to FIG. 3, illustrated is a cross-sectional view of a typical air/water valve cylinder port 12 showing the placement of four internal channel openings 20a-20d. The air/water valve cylinder port is the location through which all channels of the air and water systems in the endoscope can be accessed. These channels may terminate at either the distal tip of the endoscope, or on the light guide at the other end of the endoscope. By accessing these channels at the air/water valve cylinder port, a tool may be fed to the termination of either end of the channel. However, due to the decrease in diameter of the air/water valve cylinder port as the port progresses deeper into the control handle 10, the lower three openings 20b-20d can be difficult to see from the exterior. This decrease in diameter is characteristic of many air/water valve cylinder ports. Additionally, and as can be seen in FIG. 4, the air channel 20a makes a turn of approximately 180 degrees as it exits the air/water valve cylinder port 12 and heads into the insertion tube region 22 of the flexible endoscope. Further, the openings within the air/water valve cylinder port are not clearly visible from the exterior of the air/water valve cylinder port 12, and the channel segments may take immediate turns that are difficult to navigate without providing external support for the tool inside of the air/water cylinder port 12. Pre-bending and performing multiple attempts to introduce the tool can be time consuming and cumbersome.

A positioning aid in accordance with the invention is affixed to the control handle 10 at the endoscope air/water and suction valve cylinder ports 12, 14 to enable easy insertion of a tool into the channels of endoscope. More particularly, the positioning aid provides endoscope model-specific passageways to guide a tool into the air, water and/or balloon channel segments of the ports. The input tool openings on the positioning aid are situated to allow easy access and do not require pre-bending of the tool. As a result, the tool can be successfully introduced into channel openings of the air/water and suction valve cylinder ports on the first attempt, making the tool use easy and rapid. The positioning aid is affixed into the air/water and suction valve cylinder port in a singular orientation so that the pathway exits of the positioning aid (i.e., the locations where the positioning aid pathways end on the side wall of the positioning aid) are properly aligned with the channel segment openings of the air/water and suction valve cylinder ports 12, 14, e.g., the positioning aid can only be attached to the air/water and suction valve cylinder ports in one orientation.

Referring to FIGS. 5A-5C, illustrated is a positioning aid 50 in accordance with a first embodiment of the invention. The positioning aid 50 is attachable to an endoscope control handle 10 having air/water and suction valve cylinder ports 12, 14, and is operable to guide a tool into one or more channels of the air/water cylinder port 12. The positioning aid 50 includes a main body 52 that is couplable to the endoscope control handle 10 by inserting the positioning aid 50 into the air/water valve cylinder port 12. In the illustrated embodiment the body 52 is formed as a cylindrical structure having an open center section (i.e., a tubular structure), although a solid body is contemplated. The body 52 includes at least one guide port 53 and preferably multiple guide ports for guiding a tool into the channels of the air/water valve cylinder port 12 located in the endoscope handle 10 (FIGS. 5A and 5B illustrate three guide ports). Each guide port 53 includes an input port 54 for receiving a tool to be inserted into the air/water 12, and an output port 56 configured to correspond with one channel of the air/water or suction valve cylinder ports 12, 14. A guide pathway 58 is coupled between the input port 54 and the output port 56 and is operative to guide a tool from the input port 54 to the output port 56. In the illustrated embodiment, the input port 54, output port 56 and guide pathway 58 are formed by a flexible tube having openings at each end.

In the embodiment of FIGS. 5A and 5B, at least a portion of the guide port 53 (e.g., the input port 54 and at least part of the guide pathway 58) is formed from flexible tubing that extends out from the body 52. The flexible tubing may be formed from polytetrafluoroethylene or other material that is flexible and has a low coefficient of friction. Portions of the guide port 53 may be formed such that a transition of the guide pathway between the input port and the output port exhibits curvature in more than one plane. For example, the guide port 53 may have a helical shape. A terminus of the guide pathway 58 is fixed to an inner sidewall of the body 52 at the output port 56. A coupler 60, such as a brass or plastic tube insert or the like, connects the flexible tubing guide pathway 58 to the output port 56, as shown in FIG. 5C.

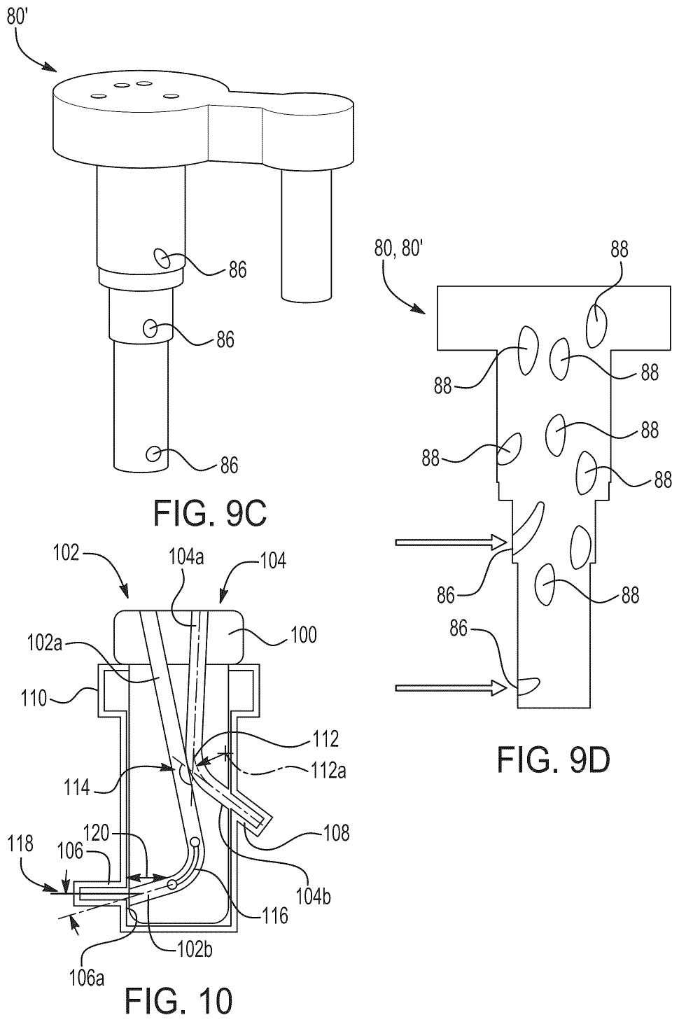

The body 52 includes a flange 52a, which operates as a mechanical stop that limits a depth of insertion of the body 52 into the valve cylinder port 12 and/or into the handle 10. By limiting the depth of insertion of the body 52, the terminus of each pathway (i.e., the output port 56) is fixed at the correct depth in the ports 12. While this embodiment of the positioning aid does not show a means of fixing the rotational position of the positioning aid, a method such as the one used in FIGS. 7 and 9C would be used.

To help in associating a guide port 54 to a channel within the endoscope ports, labels may be attached to each guide port 53 or the guide ports 53 may be color-coded, where different colors correspond to different channel segments of the endoscope. Additionally or alternatively, labels and/or color-coding may identify a size of the corresponding guide channel within the endoscope.

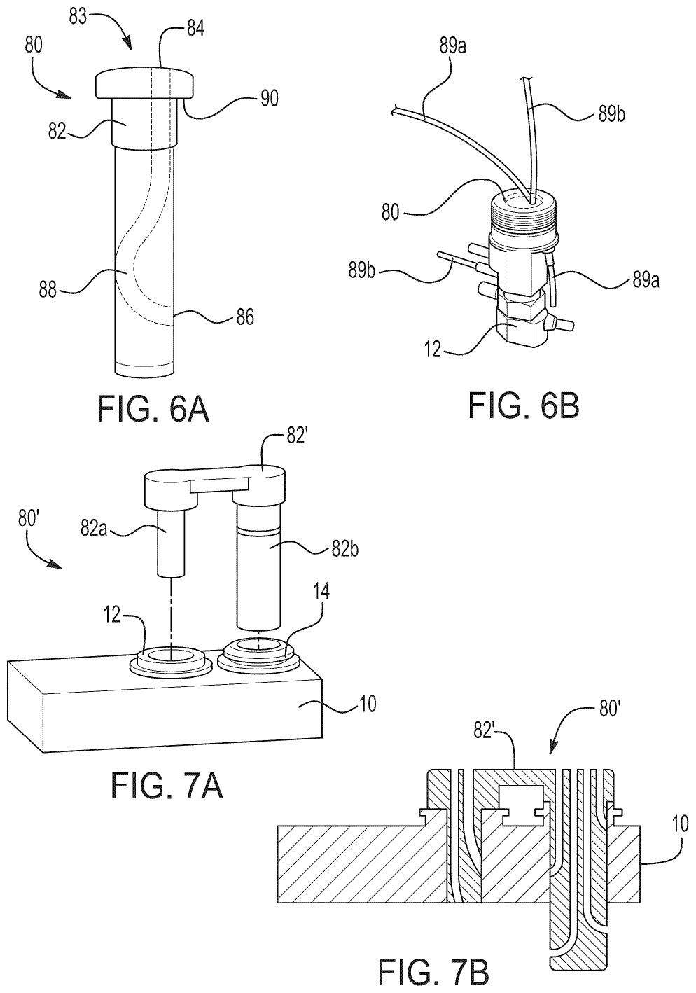

Moving now to FIGS. 6A and 6B, illustrated is another embodiment of a positioning aid 80 in accordance with the invention. FIG. 6A illustrates the positioning aid 80 without being inserted into the valve cylinder port, while FIG. 6B illustrates the positioning aid 80 inserted into an air/water valve cylinder port 12 having five channel openings. Furthermore, FIG. 6B shows how the positioning aid can be used to thread tools 89a, 89b into the channels on the air/water valve cylinder port, where the clear plastic tubing is representative of tools used to assess endoscope channel dryness, and is successfully inserted into the channels. Like the positioning aid 50 of FIGS. 5A-5C, the positioning aid 80 is attachable to an endoscope control handle 10 and is operable to guide a tool 89a, 89b into the air/water valve cylinder port 12. The positioning aid 80 includes a main body 82 that is couplable to the air/water valve cylinder port in the endoscope control handle 10 by inserting the positioning aid 80 into the air/water valve cylinder port. The body 82, which may be formed via 3D printing, injection molding, etc., includes at least one guide port 83 and preferably multiple guide ports for guiding a tool into channels of the air/water valve cylinder port 12. Like the embodiment of FIGS. 5A-5C, color-coding may be implemented to help identify the size of the guide port 83 and/or the channel in the endoscope to which the guide port is associated. Each guide port 83 includes an input port 84 for receiving a tool 89a, 89b to be inserted into the air/water cylinder port 12, and an output port 86 configured to correspond with a channel of the air/water or suction valve cylinder ports 12, 14, the input and output ports 84, 86 formed within an exterior wall of the main body 82. A guide pathway 88 couples the input port 84 to the output port 86 and is operative to guide the tool 89a, 89b from the input port 84 to the output port 86. As can be seen in FIG. 6A, the guide port 83 (i.e., the input port 84, output port 86 and guide pathway 88) is entirely formed within the body 82.

In the embodiment of FIG. 6A the guide port 83 is formed such that a transition of the guide pathway between the input port and the output port exhibits curvature in more than one plane, For example, the guide pathway may have a helical pattern. Such helical pattern is beneficial in that it gently guides a tool from the input port 84 to the output port 86 without subjecting the tool to significant bending. In this regard, an approach to the channel opening in the valve cylinder port is at a gentle angle that the tools can easily negotiate.

The body 82 also includes a flange 90 that operates as a stop to limit a depth of insertion of the body 82 into the handle/valve cylinder ports. Further, the body 82 can have multiple body portions formed as a unitary (one-piece) construction such that a single positioning aid can be simultaneously inserted into multiple valve cylinder ports. Such multi-body configuration 80′ is illustrated in FIGS. 7A and 7B. Due to the multiple body portions 82a, 82b, the body 82′ effectively forms a key or keyway that cooperates with the handle 10/valve cylinder ports 12, 14 to enable attachment of the body 82′ in only one orientation with respect to the handle 10. Different lengths and/or diameters of the body portions 82a, 82b can ensure the positioning aid 80′ may only be inserted in a specific orientation with respect to the handle 10.

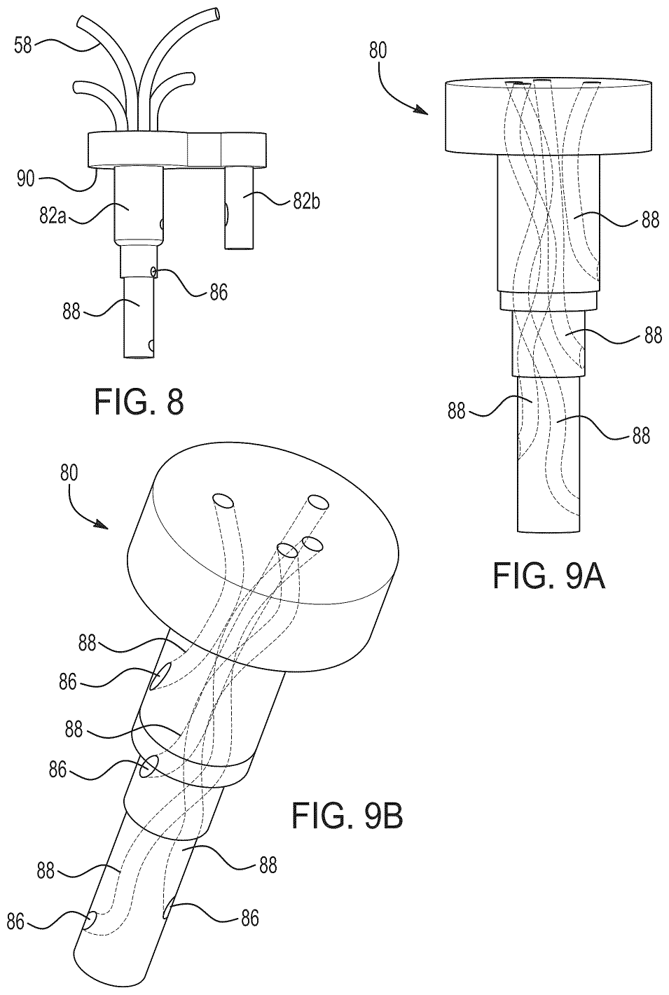

The above-discussed embodiments may be combined to form a hybrid positioning aid, whereby the flexible guide ports 53 of FIG. 5A extend out from the body 82 of FIG. 6A. Such embodiment 80′, which is illustrated in FIG. 8, would include both flexible pathways 58 and rigid pathways 88. Further, the positioning aid as described herein could be a single use device or a reusable item. When reusable, the positioning aid can be made from materials that are easy to clean and decontaminate, such as medical grade plastics and polymers.

FIGS. 9A-9B illustrate three-dimensional renderings of the positioning aid 80 of FIG. 6A, while FIG. 9C illustrates a three-dimensional rendering of the positioning aid 80′ of FIG. 7A. FIG. 9D is a sectional view of a portion of the positioning aid 80, 80′. As can be seen in the three-dimensional renderings, the guide pathways 88 have a helical approach to the output port 86. The pathways in the positioning aids seen in FIGS. 9A-9C are entirely comprised of helical curves, allowing for a smooth transition of the tool into the channel openings of the air/water valve cylinder port.

By constraining a bend angle, a bend radius and an angle of attack of the guide pathway within a defined window, and by maintaining a distance of the bend radius to an edge of the guide within prescribed guidelines, a tool inserted into the guide pathway will smoothly traverse along the guide pathway and into the channel openings. More particularly, and with reference to FIG. 10, illustrated is positioning aid 100 having two exemplary guide pathways 102 and 104, where each guide pathway corresponds to a different channel 106, 108 of the valve cylinder port 110. As seen in FIG. 10, and by way of example, the bend radius 112a is defined as the radius of a curved section 112 that joins two different segments 104a, 104b of the guide pathway 104. The bend angle 114 is defined as the angle between straight portions of the two segments 104a, 104b of the guide pathway 104 at the connection point at respective ends of the curved section 112. As will be understood, the guide pathway 102 also includes a bend radius and bend angle define by segments 102a, 102b and curved section 116.

The angle of attack 118 is defined as the counterclockwise angle between a straight portion of the guide segment 102b and the channel opening 106 of the valve cylinder port 110 as the guide pathway segment 102b approaches the channel opening 106. Finally, the distance 120 is defined as the linear (e.g., horizontal) distance between the beginning 106a of the channel opening 106 (where the channel opening is joined to the wall of the valve cylinder port) and the end point of the curved section 116 that is nearest the channel opening 106.

In order to provide smooth movement of a tool as it traverses through the guide pathway and into the channel opening, the following criteria should be observed.
The angle of attack (measured counterclockwise from the guide pathway to the channel opening) should be between 0 degrees and 30 degrees
The bend angle should be greater than 100 degrees
The bend radius should be greater than 0.1 inch.
The distance should be as large as possible.

As the tool exits the output port 86 and enters the channel within the valve cylinder port 12, 14, it is important that the guide pathway 88 and output port 86 accurately guide the tool into the channel of the valve cylinder port without becoming snagged at the interface between the output port 86 and the valve cylinder port channel. Therefore, and as noted above, an angle of attack of the guide pathway 88 and output port 86 with the corresponding channel of the valve cylinder port should be between 0 and 30 degrees. Preferably, the guide channel 88 exhibits this 0-30 degree angle at the greatest possible distance from the output port 86, but at least over the last 0.025 inches of the guide pathway 88. By making the transition from the end of the guide pathway 88 to the output port 86 relatively smooth, the likelihood that the tool will properly enter the channel of the valve cylinder port without “snagging” at the interface is significantly reduced. This constraint can also be applied to the input port 84, although since there is no interface with the valve cylinder port at the input port 84 this feature would only case of insertion of the tool into the guide pathway.

The positioning aid in accordance with the invention enables tools to be inserted within control handle valve ports of an endoscope without the need to pre-bend the tools. Further, the pathways within the positioning aid guide the tools into the channel openings within the air/water or suction cylinder ports, which makes the tools easy to insert, as the pathways in the positioning aid direct the tools into a specific hidden internal channel segment opening within the valve cylinders. Further, the pathways provide support to the tool as it negotiates entry into the channel segment.

Although the invention has been shown and described with respect to a certain embodiment or embodiments, equivalent alterations and modifications may occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein exemplary embodiment or embodiments of the invention. In addition, while a particular feature of the invention may have been described above with respect to only one or more of several embodiments, such feature may be combined with one or more other features of the other embodiments, as may be desired and advantageous for any given or particular application.

1. A positioning aid attachable to a handle of an endoscope control handle having air/water and suction valve cylinder ports having at least one channel and operable to guide a tool into at least one channel of the air/water or suction valve ports, the positioning aid comprising:
a body for coupling to the endoscope control handle, the body including at least one guide port having
an input port for receiving an instrument to be inserted into the at least one channel in the air/water or suction valve cylinder ports,
an output port configured to correspond with an opening to the at least one channel in the air/water or suction valve cylinder ports, and
a guide pathway coupled between the input port and the output port, wherein a transition of the guide pathway between the input port and the output port exhibits curvature in more than one plane.

2. A positioning aid attachable to a handle of an endoscope control handle having air/water and suction valve cylinder ports having at least one channel and operable to guide a tool into at least one of the air/water or suction valve ports, the positioning aid comprising:
a body for coupling to the endoscope control handle, the body including at least one guide port having
an input port for receiving an instrument to be inserted into the at least one channel in the air/water or suction valve cylinder ports,
an output port configured to correspond an opening to the at least one channel in the air/water or suction valve cylinder ports, and
a guide channel coupled between the input port and the output port,
wherein at least one of an angle of incidence of the guide channel at the interface with the input port or an angle of incidence of the guide channel at the interface with the output port is less than 20 degrees.

3. The positioning aid according to claim 1, wherein an angle between a channel opening of a valve cylinder port and a straight portion of a segment of the guide channel corresponding to the channel opening as the straight portion of the segment approaches the channel opening is between 0 degrees and 30 degrees.

4. The positioning aid according to claim 1, wherein a bend radius of a curved section that joins a first straight portion of a first guide channel segment of the guide channel and a second straight portion of a second guide channel segment of the guide channel is greater than 0.1 inches.

5. The positioning aid according to claim 4, wherein an angle between the first and second straight portions at a connection point at respective ends of the curved section is greater than 100 degrees.

6. The positioning aid according to claim 1, wherein the body comprises a three-dimensional printed body.

7. The positioning aid according to claim 1, wherein the at least one guide port is formed entirely within the body.

8. The positioning aid according to claim 1, wherein the guide channel is formed to have a helical pattern.

9. The positioning aid according to claim 1, wherein the input port comprises flexible tubing extending out from the body.

10. The positioning aid according to claim 9, wherein a terminus of the flexible tubing is fixed to a sidewall of the body at the output port.

11. The positioning aid according to claim 10, further comprising a coupler connecting the flexible tubing to the output port.

12. The positioning aid according to claim 11, wherein the coupler comprises a brass tube insert.

13. The positioning aid according to claim 6, wherein the flexible tubing comprises a tube formed from polytetrafluoroethylene.

14. The positioning aid according to claim 1, wherein the body comprises a keyway that cooperates with the handle to enable attachment of the body in only one orientation with respect to the handle.

15. The positioning aid according to claim 1, wherein the body comprises a stop that limits a depth of insertion of the body into the handle.

16. The positioning aid according to claim 1, wherein the guide channel is color-coded to identify a size of the guide channel.

17. The positioning aid according to claim 1, wherein the guide channel is color-coded to identify a port of the endoscope.

18. The positioning aid according to claim 2, wherein an angle between a channel opening of a valve cylinder port and a straight portion of a segment of the guide channel corresponding to the channel opening as the straight portion of the segment approaches the channel opening is between 0 degrees and 30 degrees.

19. The positioning aid according to claim 2, wherein a bend radius of a curved section that joins a first straight portion of a first guide channel segment of the guide channel and a second straight portion of a second guide channel segment of the guide channel is greater than 0.1 inches.

20. The positioning aid according to claim 2, wherein the guide channel is formed to have a helical pattern.
